(19) United States
(12) Patent Application Publication
(10) Pub. No.: US 2013/0197232 A1
(43) Pub. Date: Aug. 1, 2013

(54) PROCESS FOR THE PREPARATION OF CRYSTALLINE FORMS OF DEXLANSOPRAZOLE

(71) Applicants: Anmol Kumar Ray, Jaipur (IN); Mahavir Singh Khanna, New Delhi (IN); Rajesh Kumar Thaper, Jammu (IN); Mohan Prasad, Gurgaon (IN)

(72) Inventors: Anmol Kumar Ray, Jaipur (IN); Mahavir Singh Khanna, New Delhi (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/574,849

(22) PCT Filed: Jan. 28, 2011
(86) PCT No.: PCT/IB2011/050399

§ 371 Date: Oct. 11, 2012

(30) Foreign Application Priority Data
Jan. 29, 2010 (IN) 197/DEL/2010

(51) Int. Cl. C07D 401/12

(52) U.S. Cl. CPC C07D 401/12
USPC 546/273.7

(57) ABSTRACT

The present invention relates to crystalline forms of dexlansoprazole designated as forms A and B, and their preparation. The present invention further relates to processes for the preparation of anhydrous dexlansoprazole and dexlansoprazole sesquihydrate using crystalline Forms A and B of dexlansoprazole.

### FIELD OF THE INVENTION

The present invention relates to crystalline forms of dexlansoprazole designated as Forms A and B, and process for their preparation. The present invention further relates to processes for the preparation of anhydrous dexlansoprazole and dexlansoprazole sesquihydrate using crystalline Forms A and B of dexlansoprazole.

### BACKGROUND OF THE INVENTION

Dexlansoprazole is chemically described as 2-[(R)-{[3-methyl-4-(2,2,2-trifluoroethoxy)pyridin-2-yl]methyl}sulfinyl]-1H-benzimidazole as represented by Formula I.

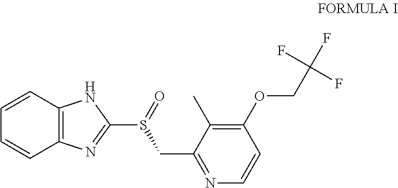

Dexlansoprazole is useful for healing all grades of erosive esophagitis (“EE”) for up to 8 weeks, to maintain the healing of EE for up to 6 months and for the treatment of heartburn associated with non-erosive gastroesophageal reflux disease (“GERD”) for 4 weeks.

U.S. Pat. Nos. 6,462,058; 7,285,668 and U.S. Publication 2007/0004779 purportedly describe processes for preparing crystalline forms of dexlansoprazole and its hydrates. WO 2009/117489 purportedly describes process for the preparation of amorphous dexlansoprazole.

### SUMMARY OF THE INVENTION

Crystalline Form A of dexlansoprazole comprising substantially the same XRPD pattern as depicted in FIG. 1.

Crystalline Form A of dexlansoprazole having an XRPD pattern comprising d-spacing values substantially at 16.18, 5.41, 4.88, 4.65, 4.15 and 3.93 Å.

Crystalline Form A of dexlansoprazole according to claim 2, further comprising d-spacing values substantially at 16.18, 13.26, 11.64, 10.88, 9.76, 8.11, 7.29, 6.76, 6.39, 5.92, 5.83, 5.73, 5.41, 5.17, 5.07, 4.88, 4.83, 4.65, 4.42, 4.27, 4.15, 4.09, 4.01, 3.93, 3.73, 3.61, 3.52, 3.45, 3.41, 3.31, 3.25, 3.21, 3.11, 3.06, 2.98, 2.92, 2.84, 2.72, 2.63, 2.55, 2.44 and 2.36 Å.

Crystalline Form B of dexlansoprazole comprising substantially the same XRPD pattern as depicted in FIG. 2.

Crystalline Form B of dexlansoprazole having an XRPD pattern comprising d-spacing values substantially at 13.86, 11.09, 5.00, 4.77, 4.62, 4.32, 3.94, 3.70 and 3.63 Å.

Crystalline Form B of dexlansoprazole according to claim 5, further comprising d-spacing values substantially at 13.86, 11.09, 9.02, 6.92, 6.80, 5.54, 5.20, 5.00, 4.77, 4.68, 4.62, 4.32, 4.29, 4.21, 4.05, 3.94, 3.70, 3.63, 3.57, 3.51, 3.43, 3.40, 3.37, 3.22, 3.09, 3.04, 2.99, 2.94, 2.89, 2.82, 2.78, 2.74, 2.67, 2.59, 2.53, 2.49 and 2.41 Å.

A process for the preparation of crystalline Form A of dexlansoprazole, wherein the process comprises:
a) treating dexlansoprazole with cyclohexanol; and
b) isolating the crystalline Form A of dexlansoprazole from the mixture thereof.

A process for the preparation of crystalline Form B of dexlansoprazole, wherein the process comprises:
a) treating dexlansoprazole with phenol; and
b) isolating the crystalline Form B of dexlansoprazole from the mixture thereof.

A process for the preparation of anhydrous dexlansoprazole, wherein the process comprises:
a) treating crystalline Form A or Form B of dexlansoprazole with an organic solvent; and
b) isolating anhydrous dexlansoprazole which comprises an XRPD pattern having interplanar spacing (d) values substantially at 11.70, 6.78, 5.84, 5.73, 4.43, 4.17, 4.13, 4.09, 3.94, 3.90, 3.70, 3.41 and 3.12 Å.

A process according to the claim 9, wherein the organic solvent is an alkanol, an ether or a mixture thereof.

A process for the preparation of dexlansoprazole sesquihydrate, wherein the process comprises:
a) treating crystalline Form A of dexlansoprazole with water, and
b) isolating dexlansoprazole sesquihydrate which comprises interplanar spacing (d) values substantially at 13.22, 9.61, 8.87, 8.04, 6.61, 6.00, 5.91, 5.64, 5.02, 4.51, 3.65, 3.57, 3.51 and 3.00 Å.

### BRIEF DESCRIPTION OF THE DRAWINGS

FIG. 1 depicts the X-ray powder diffraction pattern (XRPD) of the crystalline Form A of dexlansoprazole.

FIG. 1A provides the table of values for the XRPD pattern depicted in FIG. 1.

FIG. 2 depicts the X-ray powder diffraction pattern (XRPD) of the crystalline Form B of dexlansoprazole.

FIG. 2A provides the table of values for the XRPD pattern depicted in FIG. 2.

FIG. 3 depicts the X-ray powder diffraction pattern (XRPD) of the anhydrous dexlansoprazole.

FIG. 3A provides the table of values for the XRPD pattern depicted in FIG. 3.

FIG. 4 depicts the X-ray powder diffraction pattern (XRPD) of dexlansoprazole sesquihydrate.

FIG. 4A provides the table of values for the XRPD pattern depicted in FIG. 4.

### DETAILED DESCRIPTION OF THE INVENTION

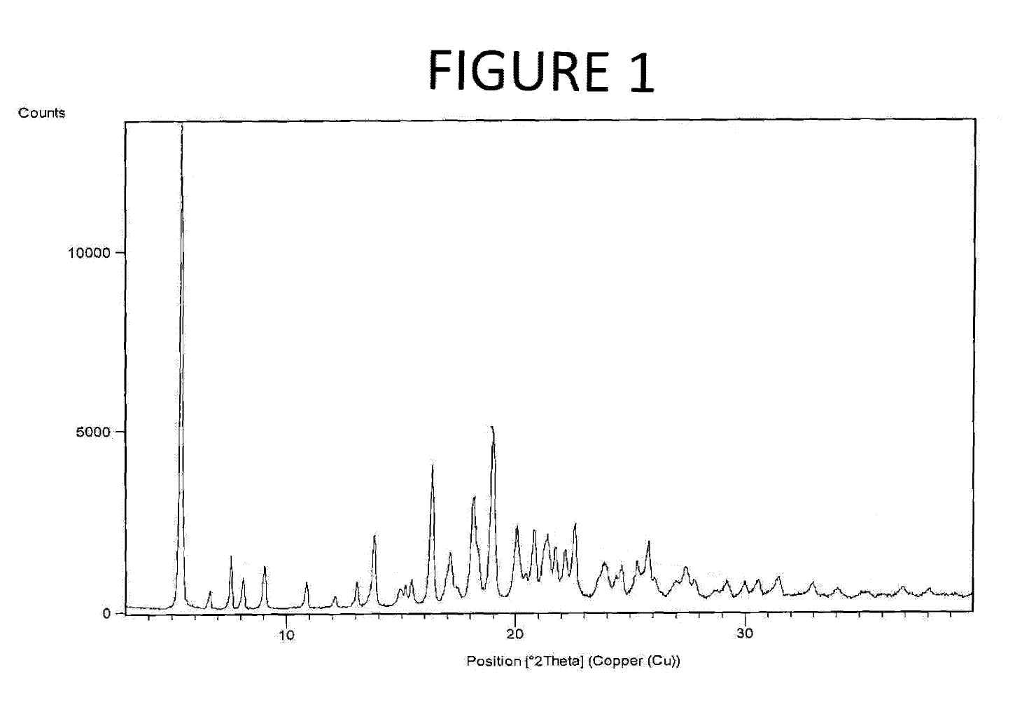

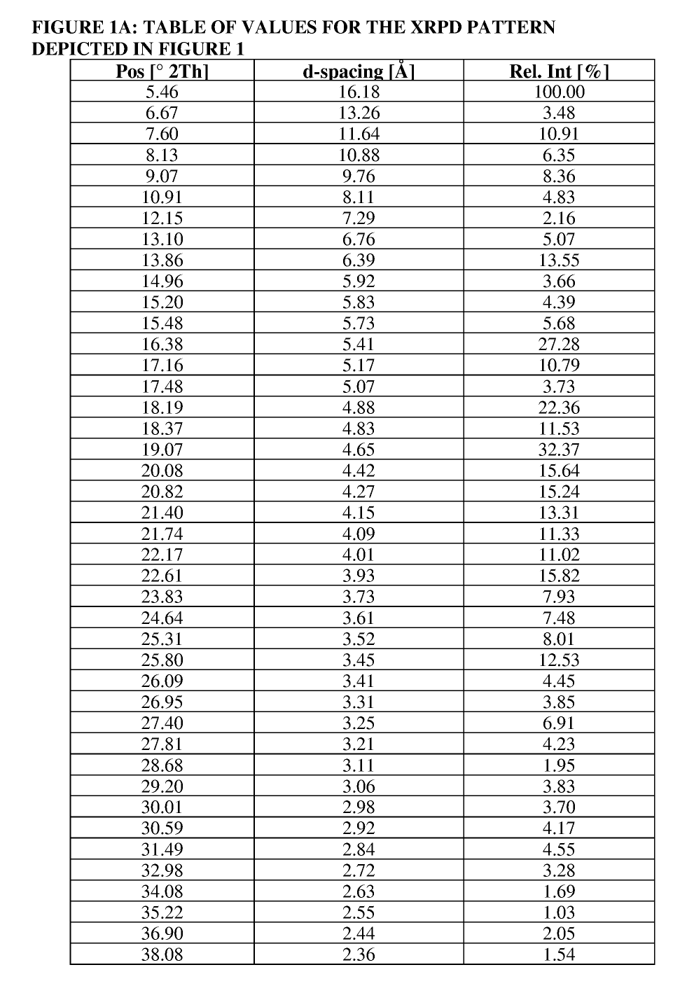

The present invention provides for crystalline Form A of dexlansoprazole. The crystalline Form A of dexlansoprazole has substantially the same XRPD (X-ray powder diffraction pattern) pattern as depicted in FIG. 1. The crystalline Form A of dexlansoprazole is characterized by an XRPD pattern having interplanar spacing (d) values at 16.18, 5.41, 4.88, 4.65, 4.15 and 3.93 Å. The crystalline Form A of dexlansoprazole is further characterized by an XRPD pattern having interplanar spacing (d) values at 16.18, 13.26, 11.64, 10.88, 9.76, 8.11, 7.29, 6.76, 6.39, 5.92, 5.83, 5.73, 5.41, 5.17, 5.07, 4.88, 4.83, 4.65, 4.42, 4.27, 4.15, 4.09, 4.01, 3.93, 3.73, 3.61, 3.52, 3.45, 3.41, 3.31, 3.25, 3.21, 3.11, 3.06, 2.98, 2.92, 2.84, 2.72, 2.63, 2.55, 2.44 and 2.36 Å.

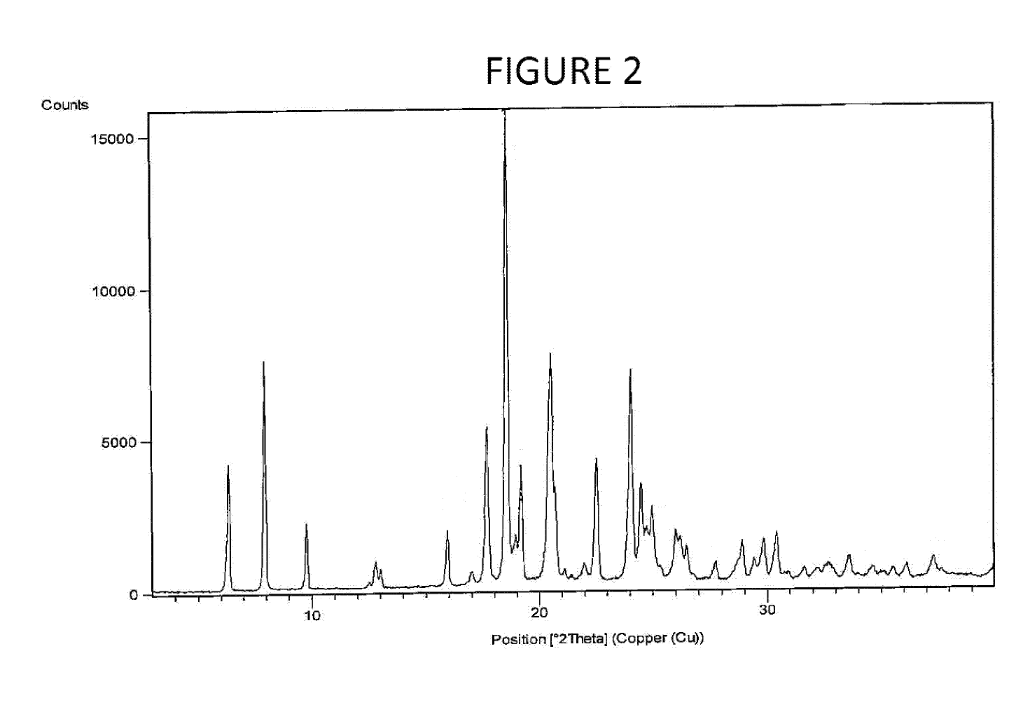

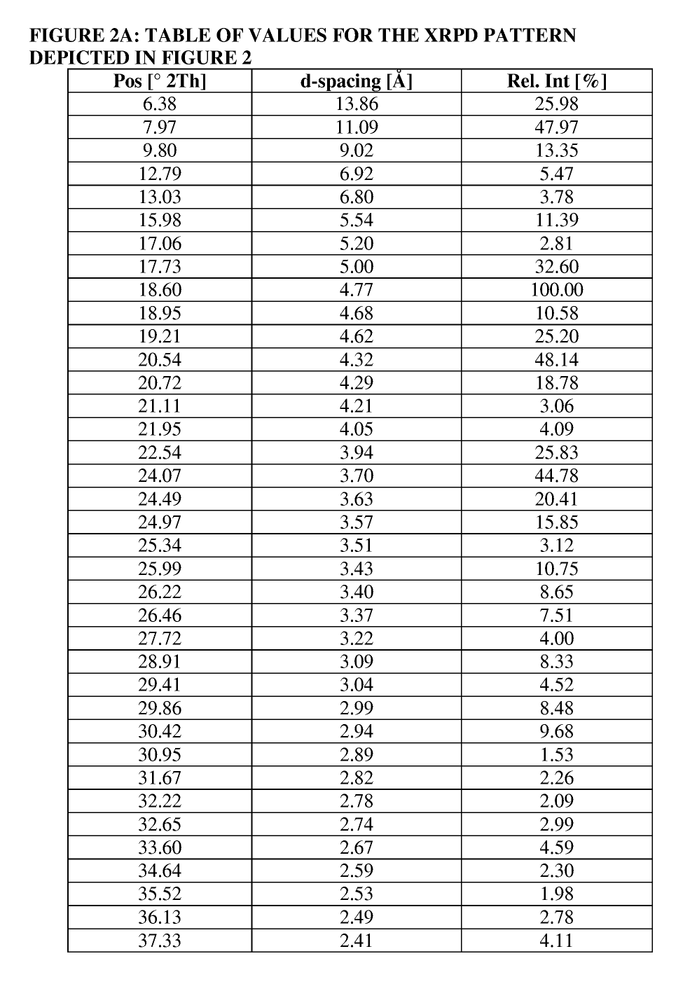

The present invention also provides for crystalline Form B of dexlansoprazole. The crystalline Form B of dexlansoprazole has substantially the same XRPD (X-ray powder diffraction pattern) pattern as depicted in FIG. 2. The crystalline Form B of dexlansoprazole is characterized by an XRPD pattern having interplanar spacing (d) values at 13.86, 11.09, 5.00, 4.77, 4.62, 4.32, 3.94, 3.70 and 3.63 Å. The crystalline Form B of dexlansoprazole is further characterized by an XRPD pattern having interplanar spacing (d) values at 13.86, 11.09, 9.02, 6.92, 6.80, 5.54, 5.20, 5.00, 4.77, 4.68, 4.62, 4.32, 4.29, 4.21, 4.05, 3.94, 3.70, 3.63, 3.57, 3.51, 3.43, 3.40, 3.37, 3.22, 3.09, 3.04, 2.99, 2.94, 2.89, 2.82, 2.78, 2.74, 2.67, 2.59, 2.53, 2.49 and 2.41 Å.

The present invention also provides a process for the preparation of crystalline Form A of dexlansoprazole. The process includes:
a) treating dexlansoprazole with cyclohexanol; and
b) isolating the crystalline Form A of dexlansoprazole from the mixture thereof.

Dexlansoprazole existing in any solid or non-solid form known in the prior art may be used as the starting material. Dexlansoprazole may be prepared, for example, according to the methods disclosed in WO 96/02535 or WO 97/02261. Dexlansoprazole is treated with cyclohexanol. The treatment with cyclohexanol may be carried out at a temperature of about 10° C. to about 100° C., for example, at about 15° C. to about 50° C. The cyclohexanol may be used alone or in combination with an organic solvent, or the treatment with cyclohexanol is optionally preceded or followed by treatment with an organic solvent. The organic solvent may be a ketone, for example, acetone, or an aliphatic hydrocarbon, for example, n-hexane. The mixture is stirred for a sufficient to time to effect the formation of crystalline Form A of dexlansoprazole. The crystalline Form A of dexlansoprazole may be isolated from the reaction mixture by filtration, cooling, evaporation, decantation, distillation, vacuum drying, or a combination thereof.

The present invention also provides a process for the preparation of crystalline Form B of dexlansoprazole. The process includes:
a) treating dexlansoprazole with phenol; and
b) isolating the crystalline Form B of dexlansoprazole from the mixture thereof.

Dexlansoprazole existing in any solid or non-solid form known in the prior art may be used as the starting material. Dexlansoprazole may be prepared, for example, according to the methods disclosed in WO 96/02535 or WO 97/02261. Dexlansoprazole is treated with phenol. The treatment with phenol may be carried out at a temperature of about 10° C. to about 100° C., for example, about 15° C. to about 50° C. The phenol may be used alone or in combination with an organic solvent, or the treatment with phenol is optionally preceded or followed by treatment with an organic solvent. The organic solvent may be an ester, for example, ethyl acetate, or an aliphatic hydrocarbon, for example, n-hexane. The mixture is stirred for a sufficient to time to effect the formation of crystalline Form B of dexlansoprazole. The crystalline Form B of dexlansoprazole may be isolated from the reaction mixture by filtration, cooling, evaporation, decantation, distillation, vacuum drying, or a combination thereof.

The present invention also provides for a process for the preparation of anhydrous dexlansoprazole which is characterized by an XRPD pattern comprising interplanar spacing (d) values at 11.70, 6.78, 5.84, 5.73, 4.43, 4.17, 4.13, 4.09, 3.94, 3.90, 3.70, 3.41 and 3.12 Å. The process includes:
a) treating crystalline Form A or Form B of dexlansoprazole with an organic solvent; and
b) isolating anhydrous dexlansoprazole which is characterized by an XRPD pattern having interplanar spacing (d) values substantially at 11.70, 6.78, 5.84, 5.73, 4.43, 4.17, 4.13, 4.09, 3.94, 3.90, 3.70, 3.41 and 3.12 Å.

The crystalline Form A or Form B of dexlansoprazole may be prepared according to the previous aspects of the present invention. The crystalline Form A or Form B of dexlansoprazole is treated with an organic solvent. The organic solvent may be an alkanol, for example, methanol, or ether, for example, diisopropyl ether or a mixture thereof. The treatment with the solvent may be carried out at a temperature of about −30° C. to about 60° C., for example, about −20° C. to about 55° C. The mixture may be stirred for about 1 hour to about 10 hours. The anhydrous dexlansoprazole may be isolated by filtration, distillation, decantation, vacuum drying, evaporation, or a combination thereof.

The anhydrous dexlansoprazole so obtained has an XRPD pattern having interplanar spacing (d) values at 11.70, 6.78, 5.84, 5.73, 4.43, 4.17, 4.13, 4.09, 3.94, 3.90, 3.70, 3.41 and 3.12 Å. The anhydrous dexlansoprazole is further characterized by an XRPD pattern having interplanar spacing (d) values at 11.70, 8.47, 7.96, 6.78, 6.53, 5.84, 5.73, 5.12, 4.85, 4.78, 4.43, 4.40, 4.24, 4.17, 4.13, 4.09, 3.98, 3.94, 3.90, 3.85, 3.70, 3.52, 3.41, 3.39, 3.31, 3.26, 3.12, 3.10, 2.97, 2.94, 2.87, 2.84, 2.75, 2.71, 2.60, 2.48, 2.41, 2.38 and 2.30 Å.

The present invention also provides a process for the preparation of dexlansoprazole sesquihydrate which is characterized by an XRPD pattern with interplanar spacing (d) values at 13.22, 9.61, 8.87, 8.04, 6.61, 6.00, 5.91, 5.64, 5.02, 4.51, 3.65, 3.57, 3.51 and 3.00 Å, wherein the process includes:
a) treating crystalline Form A of dexlansoprazole with water; and
b) isolating dexlansoprazole sesquihydrate.

The crystalline Form A of dexlansoprazole may be prepared according to the previous aspect of the present invention. The crystalline Form A of dexlansoprazole is treated with water. The water may be used alone or in combination with a water-miscible organic solvent. The treatment with water may be carried out at a temperature of about 15° C. to about 100° C., for example, about 20° C. to about 55° C. accompanied by stirring. The stirring may be carried out for about 1 hour to about 5 hours, for example, about 2 hour to 3 hours. The dexlansoprazole sesquihydrate may be isolated by filtration, cooling, distillation, decantation, vacuum drying, evaporation, or a combination thereof. The dexlansoprazole sesquihydrate so obtained has an XRPD pattern having interplanar spacing (d) values at 13.22, 9.61, 8.87, 8.04, 6.61, 6.00, 5.91, 5.64, 5.02, 4.51, 3.65, 3.57, 3.51 and 3.00 Å. The dexlansoprazole sesquihydrate is further characterized by an XRPD pattern having interplanar spacing (d) values at 21.60, 18.04, 13.22, 10.74, 9.61, 8.87, 8.04, 7.15, 6.61, 6.00, 5.91, 5.64, 5.45, 5.02, 4.80, 4.68, 4.51, 4.38, 4.29, 4.23, 4.12, 3.99, 3.85, 3.75, 3.72, 3.65, 3.57, 3.51, 3.47, 3.40, 3.34, 3.28, 3.20, 3.17, 3.11, 3.08, 3.00, 2.88, 2.83, 2.74, 2.66, 2.61, 2.55, 2.50, 2.43 and 2.40 Å.

XRPD of the samples were determined by using Panalytical X'Pert Pro X-Ray Powder Diffractometer in the range 3-40 degree 2 theta and under tube voltage and current of 45 Kv and 40 mA respectively. Copper radiation of wavelength 1.54 angstrom and Xceletor detector were used.

While the present invention has been described in terms of its specific embodiments, certain modifications and equivalents will be apparent to those skilled in the art and are intended to be included within the scope of the present invention.

### EXAMPLES

### Example 1

### Preparation of Crystalline Form A of Dexlansoprazole

Dexlansoprazole (15 g) was dissolved in cyclohexanol (120 mL) at 22° C. to 28° C. The solution was stirred at 22° C. to 28° C. for 15 minutes and at 50° C. to 55° C. for 1 hour. n-Hexane (300 mL) was slowly added at 50° C. to 55° C. in 45 minutes. The solution was stirred at 50° C. to 55° C. for 30 minutes and at 22° C. to 28° C. for 45 minutes. n-Hexane (200 mL) was added at 22° C. to 28° C. and stirred for 2 hours. The mixture was stirred at 10° C. to 15° C. for 30 minutes and at 5° C. to 10° C. for 2 hours. The mixture was filtered and the solid was washed with n-hexane (240 mL). The solid was dried under vacuum at 22° C. to 28° C. for 1 hour to obtain the title compound having an XRPD pattern as depicted in FIG. 1.

Yield: 15 g

### Example 2

### Preparation of Crystalline Form A of Dexlansoprazole

Cyclohexanol (0.8 g) and acetone (1 mL) were added to dexlansoprazole (2 g) and the mixture was ground at 22° C. to 28° C. for 20 minutes. The mixture was dried under vacuum at 22° C. to 28° C. for 32 hours to obtain the title compound.

Yield: 2 g

### Example 3

### Preparation of Crystalline Form B of Dexlansoprazole

Dexlansoprazole (2 g) was dissolved in ethyl acetate (15 mL) at 22° C. to 28° C. The solution was stirred at 22° C. to 28° C. for 10 minutes. Phenol (1.25 g) in ethylacetate (5 mL) was added drop-wise to the solution. The mixture was stirred at 22° C. to 28° C. for 2.5 hours. n-Hexane (35 mL) was added at 20° C. to 25° C. and the mixture was stirred for 7 hours. The solution was kept at 22° C. to 28° C. for 16 hours. The solution was filtered and dried under vacuum at 22° C. to 28° C. for 1 hour to obtain the title compound having an XRPD pattern as depicted in FIG. 2.

Yield: 1 g

### Example 4

### Preparation of Crystalline Form B of Dexlansoprazole

A mixture of dexlansoprazole (2 g) and phenol (1.3 g) was ground at 22° C. to 28° C. for 30 minutes. The mixture was dried under vacuum at 22° C. to 28° C. for 20 hours to obtain the title compound.

Yield: 2.5 g

### Example 5

### Preparation of Anhydrous Dexlansoprazole

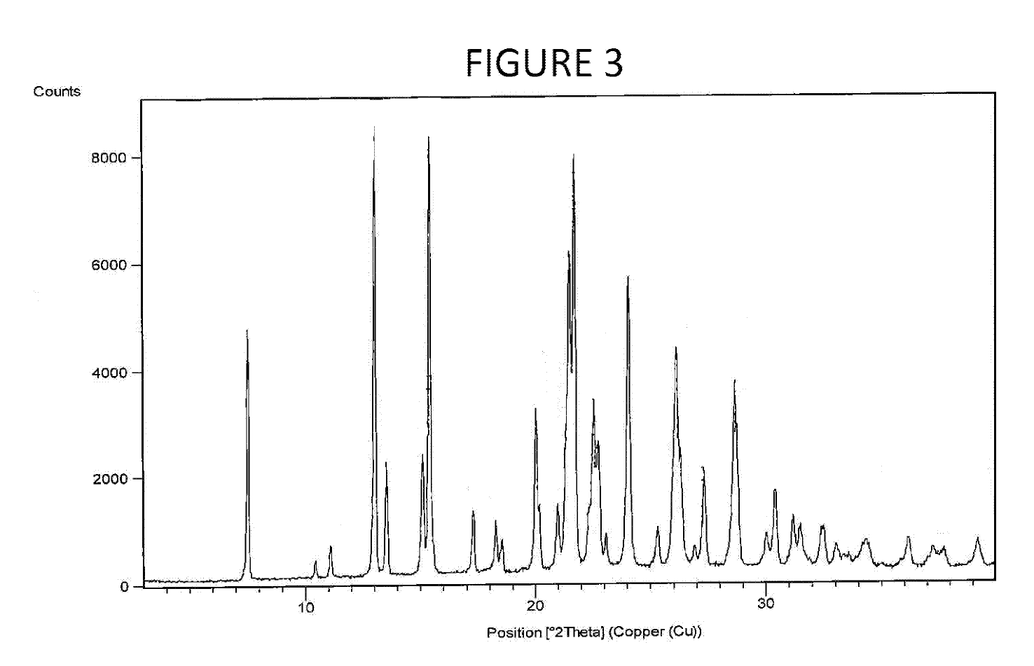

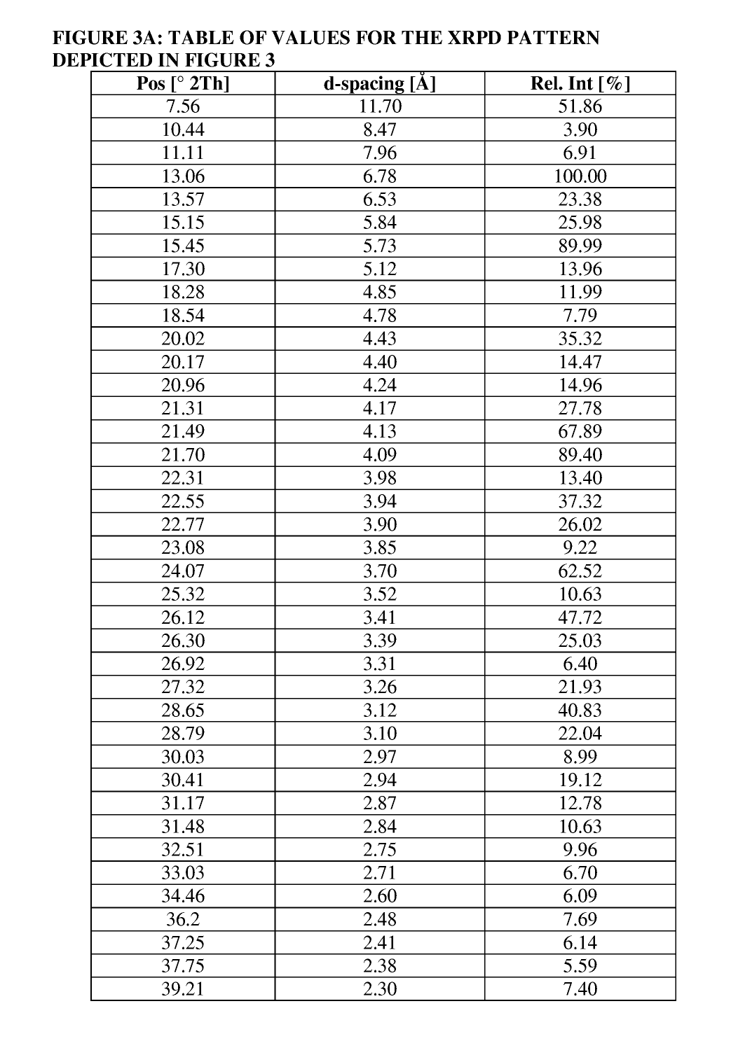

Dexlansoprazole crystalline Form A (5 g) was dissolved in methanol (12.5 mL) at 22° C. to 28° C. and the solution was stirred for 1 hour. The solution was added drop-wise in 15 minutes to cooled diisopropylether (0° C. to 5° C.; 150 mL). The mixture was stirred at 0° C. to 5° C. for 2 hours and at −20° C. to −10° C. for 45 minutes. The mixture was further stirred at 50° C. to 55° C. for 45 minutes and at 0° C. to 5° C. for 2.5 hours. The mixture was filtered and the solid was washed with n-hexane (25 mL). The mixture was dried under vacuum at 22° C. to 28° C. for 2.5 hours to obtain the title compound having an XRPD pattern as depicted in FIG. 3.

Yield: 2 g

Moisture content: 0.13% w/w

### Example 6

### Preparation of Anhydrous Dexlansoprazole

Dexlansoprazole crystalline Form B (3.8 g) was dissolved in methanol (10 mL) at 22° C. to 28° C. and the solution was stirred for 1 hour. The solution was added drop-wise in 15 minutes to hot diisopropylether (50° C. to 55° C.; 100 mL). The mixture was stirred at 50° C. to 55° C. for 1 hour and stirred at 0° C. to 5° C. for 5 hours. Diisopropylether (20 mL) was added to the mixture and the mixture was stirred for 2 hours. n-Hexane (150 ml) was added to the mixture and the mixture was stirred at 0° C. to 5° C. for 1.5 hours. The mixture was filtered and the solid was washed with n-hexane (25 mL). The solid was dried under vacuum at 22° C. to 28° C. for 2.5 hours to obtain the title compound.

Yield: 2 g

Moisture content: 0.13% w/w

### Example 7

### Preparation of Dexlansoprazole Sesquihydrate

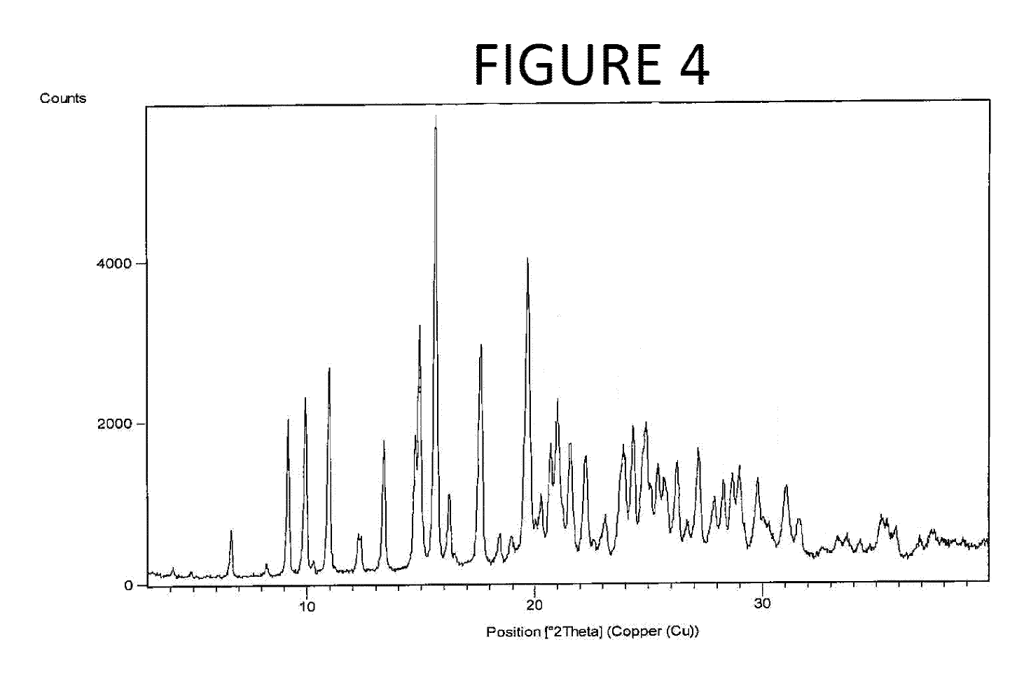

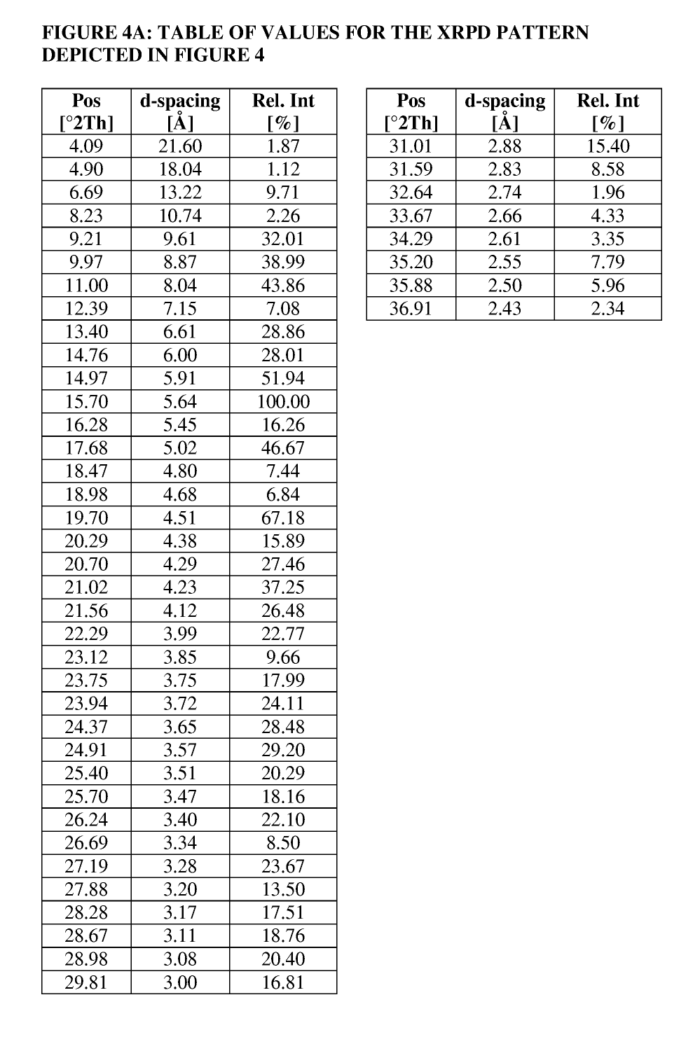

Dexlansoprazole crystalline Form A (4 g) was dissolved in deionized water (100 mL) at 22° C. to 28° C. and stirred at 50° C. to 55° C. for 30 minutes. Deionized water (50 mL) was added to the solution and the solution was stirred at 50° C. to 55° C. for 45 minutes. The mixture so obtained was filtered and the solid was washed with warm water (25 mL). The solid was dried under vacuum at 22° C. to 28° C. for 16 hours to obtain the title compound having an XRPD pattern as depicted in FIG. 4.

Yield: 3.5 g

Moisture content: 7.46% w/w

We claim:

1. Crystalline Form A of dexlansoprazole comprising substantially the same XRPD pattern as depicted in FIG. 1.

2. Crystalline Form A of dexlansoprazole having an XRPD pattern comprising d-spacing values substantially at 16.18, 5.41, 4.88, 4.65, 4.15 and 3.93 Å.

3. Crystalline Form A of dexlansoprazole according to claim 2, further comprising d-spacing values substantially at 16.18, 13.26, 11.64, 10.88, 9.76, 8.11, 7.29, 6.76, 6.39, 5.92, 5.83, 5.73, 5.41, 5.17, 5.07, 4.88, 4.83, 4.65, 4.42, 4.27, 4.15, 4.09, 4.01, 3.93, 3.73, 3.61, 3.52, 3.45, 3.41, 3.31, 3.25, 3.21, 3.11, 3.06, 2.98, 2.92, 2.84, 2.72, 2.63, 2.55, 2.44 and 2.36 Å.

4. Crystalline Form B of dexlansoprazole comprising substantially the same XRPD pattern as depicted in FIG. 2.

5. Crystalline Form B of dexlansoprazole having an XRPD pattern comprising d-spacing values substantially at 13.86, 11.09, 5.00, 4.77, 4.62, 4.32, 3.94, 3.70 and 3.63 Å.

6. Crystalline Form B of dexlansoprazole according to claim 5, further comprising d-spacing values substantially at 13.86, 11.09, 9.02, 6.92, 6.80, 5.54, 5.20, 5.00, 4.77, 4.68, 4.62, 4.32, 4.29, 4.21, 4.05, 3.94, 3.70, 3.63, 3.57, 3.51, 3.43, 3.40, 3.37, 3.22, 3.09, 3.04, 2.99, 2.94, 2.89, 2.82, 2.78, 2.74, 2.67, 2.59, 2.53, 2.49 and 2.41 Å.

7. A process for the preparation of crystalline Form A of dexlansoprazole, wherein the process comprises:
a) treating dexlansoprazole with cyclohexanol; and
b) isolating the crystalline Form A of dexlansoprazole from the mixture thereof.

8. A process for the preparation of crystalline Form B of dexlansoprazole, wherein the process comprises:
a) treating dexlansoprazole with phenol; and
b) isolating the crystalline Form B of dexlansoprazole from the mixture thereof.

9. A process for the preparation of anhydrous dexlansoprazole, wherein the process comprises:
a) treating crystalline Form A or Form B of dexlansoprazole with an organic solvent; and
b) isolating anhydrous dexlansoprazole which comprises an XRPD pattern having interplanar spacing (d) values substantially at 11.70, 6.78, 5.84, 5.73, 4.43, 4.17, 4.13, 4.09, 3.94, 3.90, 3.70, 3.41 and 3.12 Å.

10. A process according to the claim 9, wherein the organic solvent is an alkanol, an ether or a mixture thereof.

11. A process for the preparation of dexlansoprazole sesquihydrate, wherein the process comprises:
a) treating crystalline Form A of dexlansoprazole with water, and
b) isolating dexlansoprazole sesquihydrate which comprises interplanar spacing (d) values substantially at 13.22, 9.61, 8.87, 8.04, 6.61, 6.00, 5.91, 5.64, 5.02, 4.51, 3.65, 3.57, 3.51 and 3.00 Å.
